(19) United States
(12) Patent Application Publication
(10) Pub. No.: US 2012/0197061 A1
(43) Pub. Date: Aug. 2, 2012

(54) MAGNETIC COMPRESSION DELIVERY DEVICES AND RELATED DEVICES AND METHODS

(76) Inventor: Jay Anthony Requarth, Winston-Salem, NC (US)

(21) Appl. No.: 13/334,532

(22) Filed: Dec. 22, 2011

Related U.S. Application Data
(60) Provisional application No. 61/428,459, filed on Dec. 30, 2010.

(51) Int. Cl. A61N 2/10; A61N 5/00; A61N 5/10; A61N 2/00

(52) U.S. Cl. 600/2; 600/12

(57) ABSTRACT

Catheters configured to deliver magnets for magnetic compression of target tissue have at least one deflatable or retractable tissue spacer and/or radioactive material on the magnet.

### RELATED APPLICATIONS

This application claims the benefit of and priority to U.S. Provisional Application Ser. No. 61/428,459, filed Dec. 30, 2010, the contents of which are hereby incorporated by reference as if recited in full herein.

### FIELD OF THE INVENTION

This invention relates to surgical devices that may be particularly useful for forming anastomosis between two hollow viscera using magnets and/or to deliver a local intrabody therapy using cooperating magnets.

### BACKGROUND OF THE INVENTION

It is known to use magnets to create compression anastomosis for gall bladder therapy. See, U.S. Pat. No. 5,690,656 to Cope et al., the contents of which are hereby incorporated by reference as if recited in full herein. However, there remains a need for devices that can provide more accurate intrabody alignment and/or positioning of the magnets and/or that can form an anastomosis.

### SUMMARY OF EMBODIMENTS OF THE INVENTION

Embodiments of the invention are directed to devices and methods for magnetic compression anastomosis to treat patients.

Embodiments of the invention are directed to using magnetic compression to deliver spot radiation treatments to target tissue in the body.

Some embodiments are directed to catheters that include an elongate flexible catheter body and at least one magnet held in the catheter body. The catheter body also includes a window on a distal end portion of the catheter body configured to allow the magnet to exit the catheter body and at least one tissue spacer extending out from the catheter body proximate the window. The tissue spacer is configured to retract or deflate from an extended or inflated configuration.

In particular embodiments, the at least one tissue spacer is an inflatable member. The at least one tissue spacer can include a first inflatable tissue spacer proximate one end of the window and a second inflatable tissue spacer proximate an opposing longitudinal end of the window.

The at least one magnet is a rare earth magnet and may include neodymium.

In particular embodiments, the magnet can include a radioactive material. The radioactive material may include a beta emitter having a half-life that is between about 24 to about 120 hours, typically about 48 hours. The radioactive material may include Yttrium-90.

The catheter may include a force sensor held by the catheter body proximate the window.

Other embodiments are directed to delivery systems for placing intrabody magnets for generating magnetic compression. The systems include: (a) a first catheter having a magnet held therein for delivery to a target intrabody site, the first catheter having at least one inflatable tissue spacer thereon; and (b) a second catheter having a magnet held therein for delivery to a target intrabody site. The first and second catheters can deliver respective cooperating magnets to generate magnetic compression on target tissue.

The delivery systems may optionally include at least one conduit attached to the first catheter in fluid communication with the at least one inflatable tissue spacer.

The delivery systems may include a control circuit in communication with the first catheter configured to carry out at least one of the following: (a) monitoring magnetic force data sensed by a sensor on the first and/or second catheters; (b) controlling the inflation and deflation of the at least one inflatable tissue spacer thereon; or (c) generating an alert when magnetic force is detected to be insufficient.

The first catheter can include a (longitudinally extending side) magnet exit window and spaced apart first and second inflatable tissue spacers proximate each longitudinal end of the window.

At least one of the magnets of the first and second catheter may optionally include a radioactive material.

Still other embodiments are directed to surgical magnet devices. The devices can include a rare earth magnet comprising a therapeutic agent held in a sterile package for intrabody use. The therapeutic agent can include a radioactive material (typically to deliver a desired intrabody dose of radiation to a target site).

The radioactive material can include a beta emitter. The magnet may also include a string attached thereto.

Still other embodiments are directed to methods of placing intrabody magnets for magnetic compression of tissue therebetween. The methods include: (a) inserting an elongate catheter holding at least one magnet percutaneously or via a natural lumen into a patient, the catheter having an inflatable or inflated tissue spacer; (b) deflating the tissue spacer to position a magnet exit window closer to target tissue; and (c) directing the magnet to exit the window to reside against target tissue to cooperate with a different magnet separated by tissue to apply magnetic compression to the target tissue.

Yet other embodiments are directed to methods of treating intrabody tissue with radiation. The methods include: (a) inserting a rare earth magnet comprising a radioactive material into the body; (b) placing the magnet at a target treatment site; (c) compressing tissue at the target treatment site using magnetic attraction between the placed magnet and a different magnet on an opposing side of tissue at the target treatment site; and (d) exposing the tissue to a therapeutic amount of radiation from the magnet with the radioactive material.

Particular embodiments provide devices and methods to create a therapeutic magnetic compression cholecysto-duodenal fistula (“McCDF”).

Some embodiments provide magnets with radioactive material to form the anastomosis.

The foregoing and other objects and aspects of the present invention are explained in detail in the specification set forth below.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF THE DRAWINGS

FIG. 1 is a schematic illustration of cooperating catheters for placing devices, such as magnets, in the body in alignment according to embodiments of the present invention.

FIG. 2 is a schematic illustration of the catheters shown in FIG. 1 in an exemplary use environment for creating a therapeutic magnetic compression cholecysto-duodenal fistula (“McCDF”) according to embodiments of the present invention.

FIG. 3A is a schematic illustration of alternate configurations of catheters according to embodiments of the present invention.

FIG. 3B is a schematic illustration of yet other alternate configurations of catheters according to embodiments of the present invention.

FIG. 3C is a schematic illustration of an additional configuration of a catheter according to embodiments of the present invention.

FIG. 4 is a sectional view taken along line 4-4 in FIG. 3B according to embodiments of the present invention.

FIG. 5 is a front perspective view of exemplary magnets that can be placed in the body using catheters according to embodiments of the present invention.

FIG. 6 is a front perspective view of exemplary magnets with a radioactive material according to embodiments of the present invention.

FIG. 7 is a schematic illustration of a system for delivering intrabody magnets using inflatable/deflatable spacers according to embodiments of the present invention.

FIGS. 8A-8C are schematic illustrations exemplary catheters according to embodiments of the present invention,

FIG. 9A is a schematic illustration of a catheter with a magnet held in an exit window that is deformable to release and place a magnet held according to embodiments of the present invention.

FIG. 9B is a top view of an exemplary deformable window for the catheter of FIG. 9A according to embodiments of the present invention.

FIG. 9C is a schematic side view of an inflatable member that can be used to force the magnet out of the deformable window of FIG. 9A according to embodiments of the present invention,

FIG. 10 is a schematic illustration of a catheter with at least two inflatable members and corresponding fluid paths and valves according to embodiments of the present invention.

FIGS. 11A and 11B are schematic illustrations of a distal portion of a catheter having a biasing (compressed) member that can push the magnet into a delivery window according to embodiments of the present invention,

FIG. 12 is a schematic illustration of a catheter configured to deploy more than one magnet using different delivery windows according to embodiments of the present invention.

FIG. 13 is a schematic illustration of a catheter configured to deploy more than one magnet using the same deliver window according to embodiments of the present invention,

FIGS. 14 and 15 are flow charts of exemplary operations that can be used to carry out methods according to some embodiments of the present invention,

### DESCRIPTION OF EMBODIMENTS OF THE INVENTION

The present invention will now be described more fully hereinafter with reference to the accompanying figures, in which embodiments of the invention are shown. This invention may, however, be embodied in many different forms and should not be construed as limited to the embodiments set forth herein. Like numbers refer to like elements throughout. In the figures, certain layers, components or features may be exaggerated for clarity, and broken lines illustrate optional features or operations unless specified otherwise. In addition, the sequence of operations (or steps) is not limited to the order presented in the figures and/or claims unless specifically indicated otherwise. In the drawings, the thickness of lines, layers, features, components and/or regions may be exaggerated for clarity and broken lines illustrate optional features or operations, unless specified otherwise.

The terminology used herein is for the purpose of describing particular embodiments only and is not intended to be limiting of the invention. As used herein, the singular forms, “a”, “an” and “the” are intended to include the plural forms as well, unless the context clearly indicates otherwise. It will be further understood that the terms “comprises,” “comprising,” “includes,” and/or “including” when used in this specification, specify the presence of stated features, regions, steps, operations, elements, and/or components, but do not preclude the presence or addition of one or more other features, regions, steps, operations, elements, components, and/or groups thereof.

It will be understood that when a feature, such as a layer, region or substrate, is referred to as being “on” another feature or element, it can be directly on the other feature or element or intervening features and/or elements may also be present. In contrast, when an element is referred to as being “directly on” another feature or element, there are no intervening elements present. It will also be understood that, when a feature or element is referred to as being “connected”, “attached” or “coupled” to another feature or element, it can be directly connected, attached or coupled to the other element or intervening elements may be present. In contrast, when a feature or element is referred to as being “directly connected”, “directly attached” or “directly coupled” to another element, there are no intervening elements present. Although described or shown with respect to one embodiment, the features so described or shown can apply to other embodiments.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein.

Embodiments of the invention are useful for veterinarian and human uses as well as for animal studies. That is, methods and devices provided by embodiments of the invention can be configured for any species of interest, e.g., mammalian including human, simian, mouse, rat, lagomorph, bovine, ovine, caprine, porcine, equine, feline, canine, and the like.

Embodiments of the invention may be particularly suitable as a non-cholecystectomy treatment for gallstones and/or bile duct obstructions which may be performed without requiring general anesthesia.

The term “catheter” refers to a flexible tube insertable into the body. The term “fluid” includes gases and liquids. The term “string” is used broadly and refers to a length of a biocompatible (sterile) thin flexible material formed of any suitable material or combinations of materials and may be in the form of a suture, thread, metallic and/or textile strip, filament, strand, braid or the like. The term “locking” refers to the engagement of the first and second magnets based on the magnetic attraction forces generated by the cooperating magnets.

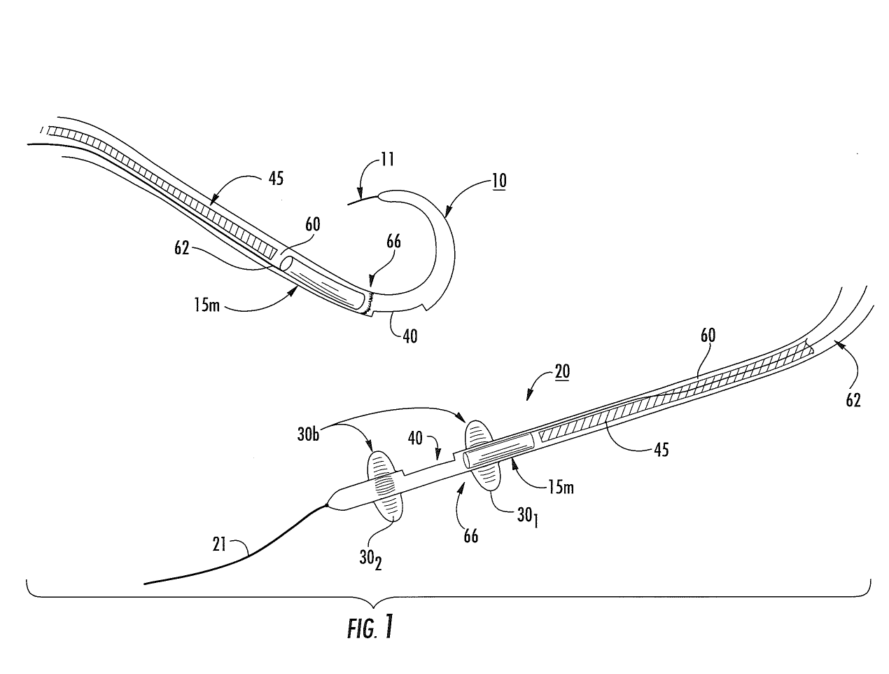

Referring now to the figures, FIGS. 1 and 2 illustrate first and second catheters 10, 20, respectively, which can be a cooperating pair of catheters that can be configured to place intrabody devices 15, typically magnets 15m, to create anastomosis between two target viscera.

However, it is noted that, in some embodiments, only one catheter may be required. Also, although catheter 10 is shown as a catheter with a deformable distal end having a guidewire 11, other catheter configurations may be used and may not include a guidewire. Similarly, although catheter 20 is shown as having a guidewire 21, it may have other configurations and be used without any guidewire.

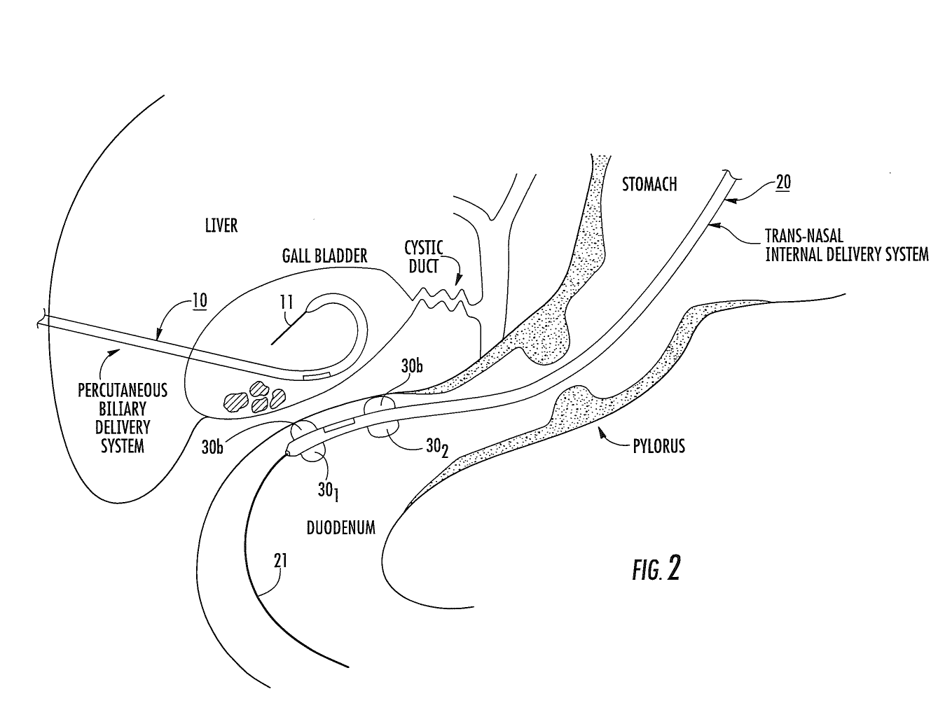

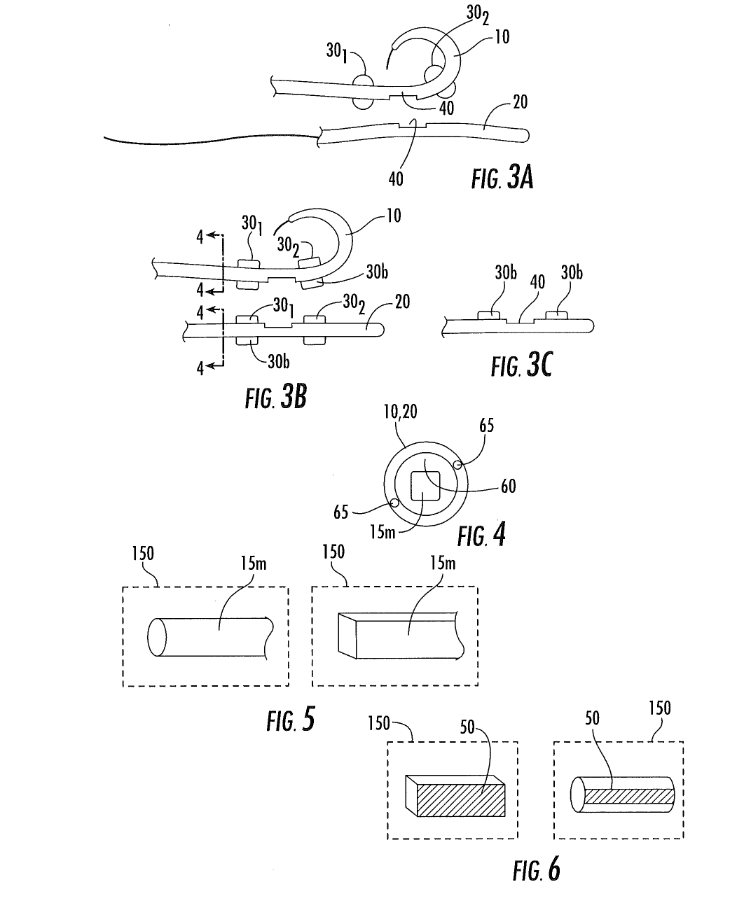

As shown in FIGS. 1 and 2, at least one catheter 10, 20 includes at least one tissue-spacer 30 to help prevent the magnet 15m from contacting local tissue until the magnet 15m is in a desired position or in a desired alignment with the magnet 15m held by the other catheter. The tissue-spacer 30 can project outwardly about an entire perimeter, e.g., the entire circumference (FIG. 1), about a portion of the perimeter (FIG. 8B), or beyond the tip of the catheter (FIG. 8C). As shown in FIG. 1, the catheter 20 can include at least two tissue-spacers 30. The tissue-spacers 30 can be configured as a pair 30₁, 30₂, one on each side of the deployment window 40, but one or more than two tissue spacers 30 may also be used. FIG. 3A illustrates that catheter 10 can have this tissue-spacer configuration while catheter 20 is devoid of any tissue-spacers. FIG. 3B illustrates that both catheters 10, 20 have the tissue spacers 30.

The tissue-spacer 30 is configured to extend a distance out beyond the bounds of the catheter body to space the magnet away from local tissue, then retract to allow the body of the catheter 10, 20 to be closer to target tissue for deployment of the magnet from the catheter window 40. The tissue spacers 30 can be a fin, finger, or other member that can space the catheter body apart from tissue, then controllably retract into or against the body of the catheter. However, in particular embodiments, the tissue-spacer 30 comprises an inflatable balloon 30b (that is also deflatable). Partial or total deflation can be used when the magnet 15m is aligned with the opposing magnet 15 across a target tissue barrier. Where more than one inflatable tissue-spacer 30b is used, they can be concurrently or independently inflated and/or deflated during use. The balloons 30b can be configured to inflate outwardly between about 1 mm to about 1 cm, typically between about 5 mm to about 50 mm, and more typically between about 10 mm to about 20 mm, to provide a corresponding amount of spacing from local tissue.

The catheters 10, 20 can be positioned in the body with the tissue-spacers 30 retracted/deflated or extended/inflated, depending on the intrabody path to the desired location. However, as a catheter 10, 20 approaches a desired location or once in the body passage that has sufficient clearance, the tissue spacer 30 can be expanded or extended. The magnet 15m can be held in the window 40 or held retracted inside the channel 60 a distance away from the window 40 during placement to the target site in the body. When the alignment and/or position is confirmed as correct or appropriate (via an imaging modality such as ultrasound and/or fluorscopy or based on force measurements of the attractant force of the two magnets), the tissue-spacer(s) 30 can be deflated and the magnet 15m deployed from the window 40, thereby positioning the magnet 15m in a desired aligned position. The inflatable/deflatable tissue-spacer 30 can have any desired inflated shape. In some embodiments, the inflatable spacer 30b can have a bulbous balloon-like shape as shown in FIG. 1 or a substantially constant annular shape (FIG. 3B). In other embodiments as shown in FIG. 3C, the inflatable tissue spacer 30b can extend about a portion of a transverse cross-section, typically on the same side as the window exit 40.

Still referring to FIGS. 1 and 2, the catheters 10, 20 can include a magnet pusher 45 or other magnet deployment member that can expel and/or movably position the magnet to exit the catheter window 40. In some embodiments, the catheter 10, 20 can include an inwardly extending shoulder 66 (e.g., crimp, resilient fins or fingers, or ledge) that acts as a stop to inhibit early deployment. The shoulder 66 can reside upstream of the window 40 but is typically proximate the window to limit actuation distance of the pusher. However, the magnet 15m can be locked at a desired position using a string or other locking configurations and a retaining shoulder is not required.

The pusher 45 is configured to push the magnet out of the catheter 10, 20. The pusher 45 can be an elongate member that can be slidably advanced in the channel 60 of the catheter 10, 20 to physically push the magnet toward the window 40 (and beyond the shoulder 66, where used). As shown in FIGS. 11A and 11B, the magnet pusher 45′ may alternatively or additionally include a biasing member 45b such as a spring or elastic compressed resilient member that can be released or unbound to force the magnet forward or rearward (the latter if held distal to the window) toward the window 40. In one embodiment, the biasing member 45b is held distal of the magnet 15m in the catheter in a compressed state (FIG. 11A) by an elongate member 46 having an arm 46a. Turning the elongate member 46 turns the arm 46a outwardly and releases a proximal end of the biasing member 45b, which expands to force the magnet 15m to enter the window 40. Pressurized fluid can also or alternatively be used to force the magnet to exit the window 40 (not shown).

FIG. 4 illustrates a section view of an exemplary catheter 10, 20. As shown, the catheter 10, 20 includes a channel 60 that can hold the magnet 15m and/or pusher 45. At least one fluid path 65 extends from a proximal end of the catheter to the inflatable balloon 30b inside the catheter body and along a length thereof (typically as a longitudinal channel fog bed through a thickness of an outer wall) of the catheter. Alternatively, the fluid path can be formed using at least one discrete tube 65 that resides in the channel 60 can be used to inflate the spacer 30b (where an inflatable version is used). The inflatable balloon spacer 30b can be inflated similar to a FOLEY catheter using any suitable fluid, typically saline. Where two (or more) paths 65 are used, one can extend to a respective balloon spacer 30b, both path, 65 can extend to both spacers 30b (where more than one spacer 30b is used) or multiple paths 65 can be used to inflate a respective spacer 30b. Alternatively, one path 65 can be an inflation path and the other can be used for deflation where the fluid is desired to remain captured and not released in the patient (at least at the magnet site). As yet another alternative, a two-way valve can be used to allow the same path to be used for inflation and deflation.

The magnet 15m can have any suitable configuration, but generally has a length dimension that is greater than the width or radius. The magnet 15m can be sterile and held in a sterile package 150 for medical use. The magnet 15m can be held in a flexible container (typically with insulators such as foam packing or other material that inhibits breakage during shipment) and/or may be included with a catheter as a kit or packaged in other suitable configurations. The term “sterile” refers to devices that meet cleanliness standards guidelines for human and/or animal surgical uses (e.g., FDA guidelines in the U.S.). FIG. 5 illustrates two exemplary shapes, a relatively thin polygon shape and a cylinder. The magnets 15m can be sized and configured to form a desired anastomosis size. They should have sufficient magnetic strength to apply sufficient compressive force to form an ischemic anastomosis (when used for this purpose). The magnets 15m can vary in length depending on the end use, but are typically between about 2-20 mm long and 1-10 mm in width or diameter. The slit (e.g., opening) formed by the magnetic compression is typically about two times the length of the magnet 15m. For example, for certain embodiments, the magnets 15m can form a relatively large hole such as between about 3 mm-20 mm across. In particular embodiments, the magnets 15m can be sized and configured to create an anastomosis of about 48 French (typically for treating patients with unresectable pancreatic cancer). In other embodiments, smaller openings can be formed, such as between about 1-2 mm (this size may be particularly suitable for the ureter).

The magnet 15m can comprise a rare earth magnet, which is typically much stronger than ferrous magnets. There are two conventional types of rare earth magnets, neodymium magnets (e.g., neodymium-iron-boron) and samarium-cobalt magnets. Rare earth magnets can be extremely brittle and can also be vulnerable to corrosion (such as from the digestive acids in the body). The magnets 15m can be plated or coated with a biocompatible material such as silicone to protect them from breaking, chipping and/or for corrosion resistance. Particular examples of rare earth magnets include Nd₂Fe₁₄B, SmCo₅ and Sm(Co,Fe,Cu,Zr)₇. However, it is contemplated that Nd₂Fe₁₄B may be particularly suitable for some embodiments.

FIGS. 1 and 2 also illustrate that at least one string 62 can be attached to the magnet 15m to allow for retrieval, in some embodiments. The string 62 can reside outside the pusher member 45 as shown or extend through an axially extending slot or channel formed on an exterior wall of the pusher or formed through a center or other interior location of the pusher body. The string 62 can be detachable from the magnet 15m, once in position in the body, or may remain on the magnet 15m. Alternatively or additionally, the string 62 may be resorbable, depending on the end use.

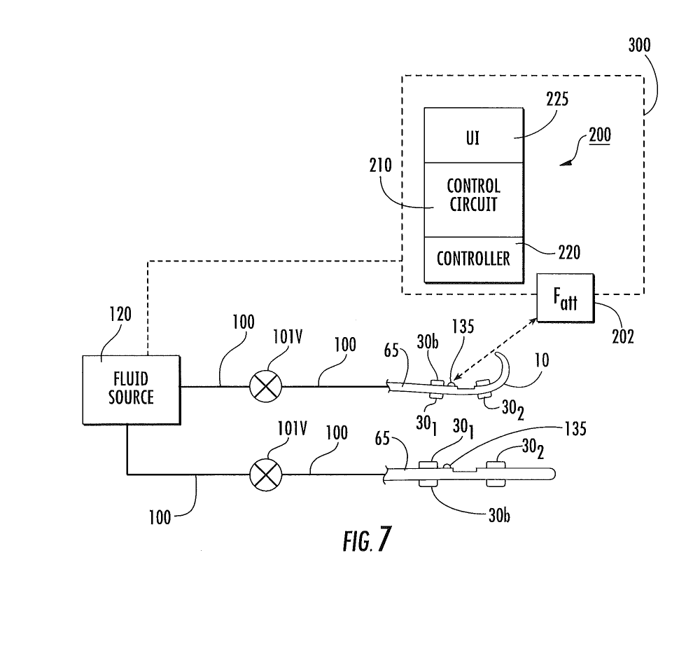

FIG. 6 illustrates that one or both magnets 15m (where two are used) can include a radioactive material 50. The term “radioactive material” refers to any suitable radioactive emitter material that can be placed onto or into the magnet or other target device. The radioactive material may provided to the magnet 15m (or substrate forming part of the magnet assembly) in any manner, such as in a laminated over-layer, a laminated sub-surface layer, a coating, sputter-deposition, and the like. The radioactive material can be a beta emitter, such as strontium-90, carbon-14, tritium, Yttrium-90 and sulfur-35. In particular embodiments, the radioactive material comprises Yttrium-90, which has a relatively small penetration depth, e.g., about 2 mm, average to about 10 mm, average (max). The radioactive material may also comprise an alpha emitter such as radium, radon, uranium, or thorium, as such may also be able to provide a small penetration depth. In some embodiments, the cooperating magnets 15m with at least one of the magnets 15m having the radioactive material 50 configured to face the other magnet 15m can generate compression and use the radiation to act as an “atomic knife” to facilitate a clean and relatively fast anastomosis. The radioactive material 50 may be selected to have a relatively short half-life, e.g., less than about two weeks (Yittrium-90 has a half-life of about 64 hours).

The material 50 can be formed on one surface integrated into a subsurface region or be placed on the entire perimeter. It is contemplated that by selectively placing the material 50 on one surface, this surface can be held enclosed in the catheter until the magnet is in a desired location, then rotated to face out the window 40 and deployed. This can limit exposure to non-target tissue. The magnet 15m can be oriented so that the side, surface or interior having the radioactive material 50 faces and is held against the tissue using the magnetic compression to create the anastomosis. The material 50 can comprise at least one therapeutic chemical or pharmaceutical agent (e.g., a cytotoxic agent, stem cells, antibiotic, anti-inflammatory, anti-scarring, or other agent), and/or radiation for treating a disease, such as, for example, cancer.

In other embodiments, the magnets 15m, at least one of which includes the radiation material 50, can be used to apply a drug or radiation therapy in vivo and internally to treat tissue, for example cancerous tissue. In this embodiment, the magnets 15m can generate sufficient strength to hold the magnets at the treatment site allowing for localized (spot) delivery of the therapeutic agent without requiring the formation of an anastomosis. One or both of the magnets 15m may be held on the respective catheter during the therapy while still providing the magnetic compression of tissue (typically for several days or weeks) or the magnets can be deployed from the catheters to provide, for example, a radiation therapy. The radiation material can deliver a therapeutically effective amount of radiation (typically to treat cancerous sites with a (localized) cytotoxic amount of radiation therapy). The magnets 15m can be of any suitable shape and size depending on the target treatment region. As described above, the radioactive material 50 may be placed on one surface or a portion of the magnet or over an entire outer surface or in an inner layer or subsurface region of the magnet device. For example, the magnet 15m can include a first layer on at least one side of a radioactive coating, followed by a layer of silicone, followed by another layer of a radioactive coating. As shown in FIG. 12, the catheter 10, 20 may also be configured to supply more than one magnet 15m for locally treating more than one spot with radiation. FIG. 12 is a schematic illustration of a catheter configured to deploy more than one magnet 15m using different delivery windows 40 and two pushers 45. FIG. 13 is a schematic illustration of a catheter configured to deploy more than one magnet 15m using the same deliver window 40 and the same pusher 45. When used with the radioactive material 50, the magnets 15m may be passed naturally out of the body (such as when an anastomosis is formed in the duodenum) or retrieved via string or other removal tool. If the magnets 15m are allowed to pass naturally, the anastomosis may be configured to form over the number of days equivalent to or greater than at least the half-life of the radioactive material in the body or for the time that is greater than the half-life, e.g., for the Y-90, with about a 64 hour half-life, the magnets can be configured to generate an opening sufficient in size to allow the passage of the magnet pair may in a time period that is between about 10 days (when the radioactive material is no longer active).

The catheter 10, 20 can have spaced apart inflatable spacers 30b about each window 40 that can be separately inflated or deflated to help place the magnets 15m in a desired location. FIG. 12 shows the more proximal inflatable spacers 30b in a deflated or partially deflated state while the more distal inflatable spacers are inflated.

The window 40 can be a laterally extending window on one side of the catheter 10, 20 (FIG. 1) or may extend substantially about the entire perimeter. Alternatively, the window 40 can be configured to have circumferentially spaced apart window panels 40a, 40b (FIG. 8A) for selective deployment of the magnet 15m from one. The window panels 40a, 40b may be axially offset from each other a defined distance instead of aligned across from each other as shown in FIG. 8A, such as between about 10%-200% of a length of the magnet 15m. In yet other embodiments, the exit window 40 may be located on an end of the catheter rather than along a side.

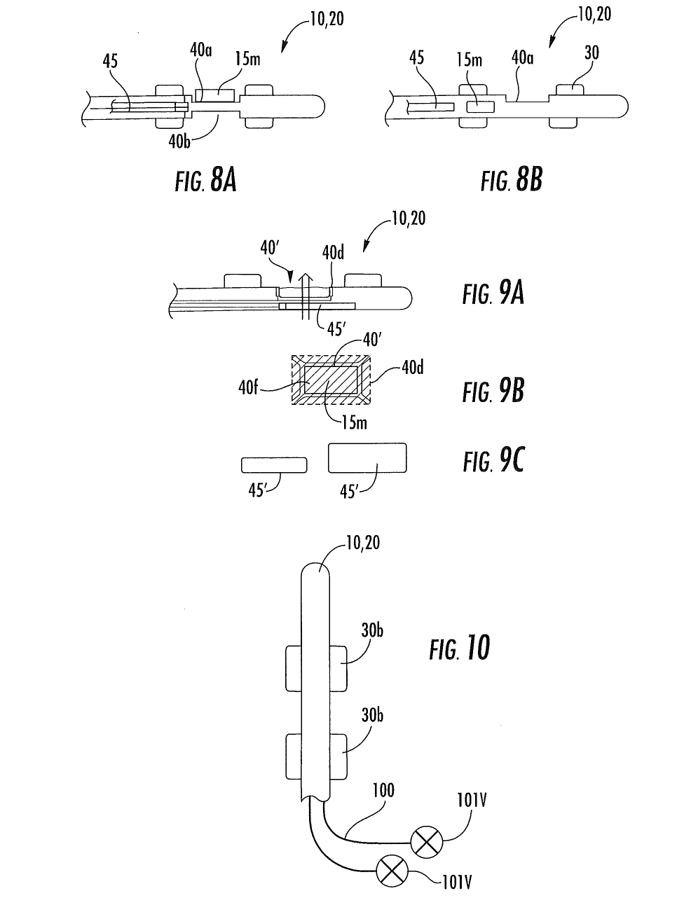

Referring to FIGS. 9A and 9B, in some embodiments, the window 40′ can have a deformable outer perimeter 40d. For example, the window 40′ can have a scored or preferentially weaker boundary that when pushed with sufficient pressure/force allows the underlying magnet to pass therethrough. The window 40′ can have a size in the non-deformed shape that is less than that of the magnet body. In this configuration, the magnet 15m may be held under the window 40 during insertion of the catheter in the body. The magnet 15m can be pushed out of the window by pushing out the deformable perimeter 40d of the window. FIG. 9C illustrates that the pusher 45′ can include an inflatable end portion that expands to push the magnet 15m out the window 40′.

In some embodiments, the window 40, 40′ can have a thin film biocompatible covering that extends over the open space used to expel or deploy the magnet 15m. The thin film covering can be a restorable, dissolvable biocompatible material that can be released from the catheter body and can be held by the face of the magnet 15m. This covering may help with corrosion issues and/or provide cushioning during deployment of the magnet 15m. The catheter(s) 10, 20, magnet 15m and/or film may include a time-released medicament such as an antibiotic, anti-inflammatory, anti-scarring, mucosal tissue promoter, and/or other agent. Thus, in some embodiments, the magnet 15m and/or one or both catheters 10, 20 can be configured to introduce a therapeutic agent. The agent can be configured to inhibit scar (e.g., collagen) formation and/or promote mucosal tissue growth about the anastomosis. The agent may comprise mucosal cells that are released to local tissue during the magnetic compression. The mucosal cells may improve patency. The catheter 10, 20 and/or magnet 15m can be configured to provide a time-released medicament such as one or more of: an anti-scarring agent, a mucosal tissue growth promoter, an antibiotic, an anti-inflammatory or other agent.

FIG. 7 is a schematic illustration of a system 200 used with one or more catheters 10, 20 with fluid paths 65 in fluid communication with the inflatable spacers 30b (both shown as having at least two 30₁, 30₂) on one end and conduits/tubing 100 on the other that connect to a fluid source 120. The fluid source and associated flow paths 100 can include one or more valves for inflating and/or deflating the spacers 30b. The system 200 can include a control circuit 210 with a controller that directs the inflation/deflation upon instructions from a user (such as a surgeon). The circuit 210 can have a User Interface 225 such as an HMI that allows the surgeon to indicate when to inflate and deflate the spacers 30b. Of course, the catheters 10, 20 may also or alternatively allow manual direction of activation controls for this purpose. The fluid source 120 can have flow meters that can control flow rates. The control circuit 200 can be hard wired to the fluid source or may wirelessly communicate with one or all the system components to direct the inflation and deflation. The control circuit 200 can be configured with a force monitoring module 202 to monitor attractant force using a force sensor 135 on one (typically both) catheters 10, 20, proximate the window 40, 40′. It is contemplated that if tissue at a local site is too thick (e.g., greater than about 1 cm) and/or the magnetic strength is not sufficiently strong, an incomplete or unsuitable anastomosis may be formed (if formed at all, at least without the radioactive material). While the tissue thickness may be measured using an imaging modality, in some embodiments, one or both catheters 10, 20 can include a force sensor 135 that also or alternatively measures a magnetic attraction force generated when the two magnets 15m are proximate to each other. This force may be measured once at least one of the two magnets 15m is in position outside a respective catheter body. In some embodiments, the force can be monitored once both magnets 15m are at least partially deployed so that they are held together to apply magnetic compression. Alternatively, the force can be measured prior to actual deployment of either magnet (such as while a spacer is at least partially inflated). The system 10 can be configured to lock release of the magnet 15m based on a threshold force required before allowing deployment. The force between the two magnets 15m depends on the strength and orientation of both magnets and the distance (tissue thickness) and direction of the magnets relative to each other. The force is sensitive to rotations of the magnets due to magnetic torque. The force on each magnet depends on its magnetic moment and the magnetic field B of the other. The force sensor 135 can be configured as a wire or any suitable sensor configuration.

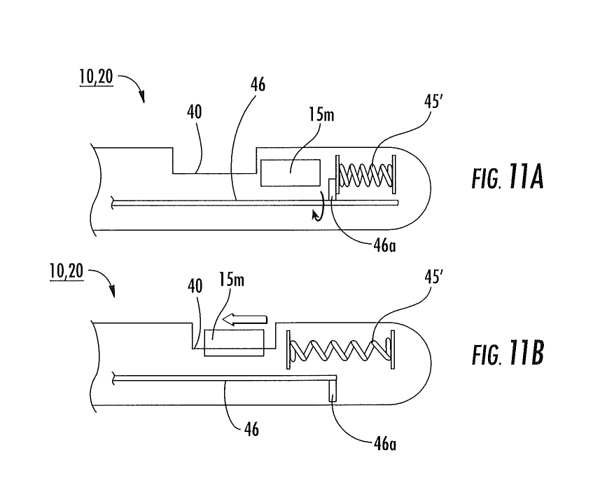

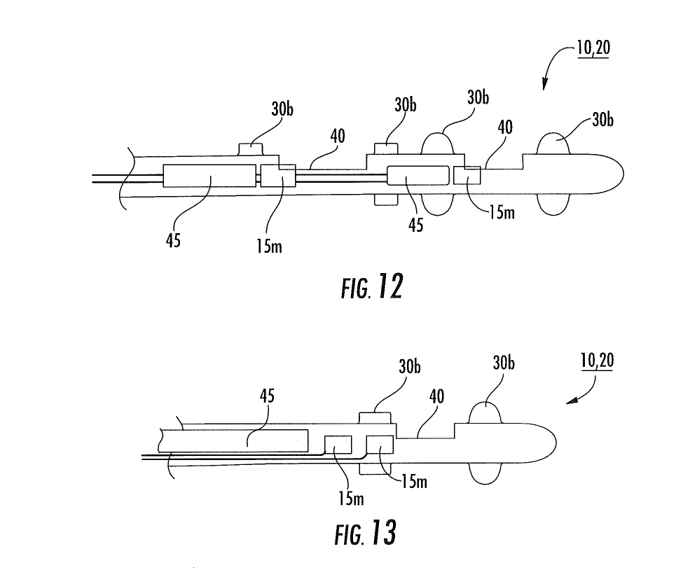

FIG. 7 illustrates one conduit 100 that is in communication with a fluid path 65 of a spacer(s) 30b. FIG. 10 is a schematic illustration of a catheter 10, 20 that can have at least two inflatable members 30b and a corresponding at least two fluid paths 65, at least two conduits 100 and valves 101 according to embodiments of the present invention.

The circuit 200 can include a digital signal processor and/or an Application Specific Integrated Circuit (ASIC) (e.g., ASIC and/or processor with software) that includes or executes part or all of the computer readable program code for generating the inflation/deflation directions, responding to input from the UI 225, and monitors force 202. The circuit 200 can include a data processing system which may, for example, be incorporated or integrated into the processor. The processor can communicate with or include electronic memory. The processor can be any commercially available or custom microprocessor. The memory is representative of the overall hierarchy of memory devices containing the software and data used to implement the functionality of the data processing system. The memory can include, but is not limited to, the following types of devices: cache, ROM, PROM, EPROM, EEPROM, flash memory, SRAM, and DRAM.

The processor or memory may include several categories of software and data used in the data processing system: the operating system; the application programs; the input/output (I/O) device drivers; and magnetic attraction force data and/or correlated tissue thickness. The latter can be used to predict if an anastomosis will likely be ischemic if the magnets are placed at the corresponding position.

As will be appreciated by those of skill in the art, the operating systems may be any operating system suitable for use with a data processing system, such as OS/2, AIX, or zOS from International Business Machines Corporation, Armonk, N.Y., Windows CE, Windows NT, Windows95, Windows98, Windows2000, WindowsXP, Windows Visa, Windows7, Windows CE or other Windows versions from Microsoft Corporation, Redmond, Wash., Palm OS, Symbian OS, Cisco IOS, VxWorks, Unix or Linux, Mac OS from Apple Computer, LabView, or proprietary operating systems. The I/O device drivers typically include software routines accessed through the operating system by the application programs to communicate with devices such as I/O data port(s), data storage and certain memory components. The application programs are illustrative of the programs that implement the various features of the data processing system and can include at least one application, which supports operations according to embodiments of the present invention. The data represents the static and dynamic data used by the application programs, the operating system, the I/O device driver and the like.

The control circuit 210 and/or UI 225 of the system 200 can wirelessly communicate with a clinician workstation 300 and/or other remote computer device or may be integrated into the clinician workstation 300. The system 200 and/or workstation 300 can include a display that can communicate with a computer which includes a portal and an Application that allows the force data to be graphically displayed for a patient record or other data record. The system or components thereof can communicate with the workstation or other remote device via a computer network including an intranet or the internet with the appropriate use of firewalls for patient privacy and compliance with HIPPA (Health Insurance Portability and Accountability Act) or other regulatory rule or authority.

The system 200 can include an electronic library of target forces correlated to separation distance, such as target force ranges per distance separation (e.g., 1 mm, 2 mm, 3 mm) that can be used to identify a desired threshold force for surgical use/referral. This can help a clinician decide whether to retrieve or deploy a magnet or change a site. The force/distance data can be generated using tissue phantoms and force curves based on magnet type, for example.

Turning again to FIG. 2, in some embodiments, the catheters 10, 20 can be used to place a respective magnets 15m for creating a linear ischemic injury in two adjacent thin-walled abdominal organs, such as the gall bladder and the duodenum and/or common bile duct and the duodenum. The catheters can be used for a bile duct decompression, to treat cholecystitis and symptomatic gallstones, for magnetic compression ischemic anastomoses, bile duct to duodenum, gall bladder to duodenum, ureter to ileal conduit, stomach to pancreatic pseudocyst, stomach to 4th portion of the duodenum, right colon to sigmoid colon, and the like. In particular embodiments, the magnets 15m can be inserted percutaeously to create an ischemic anastomosis between the gall bladder and the duodenum to palliate obstructive jaundice in patients with cancer, benign disease, or having other bile duct obstructions, without requiring the use of endo-biliary plastic drains or self-expanding stents which have a tendency to become occluded. This procedure can be a minimally invasive procedure and/or provide a cost effective treatment option.

In some embodiments, the first catheter 10 can be inserted percutaneously via the biliary (liver) into the gall bladder using conventional needle-guidewire-dilator techniques and local anesthesia. The second catheter 20 can be inserted into the duodenum via a trans-nasal-guidewire delivery system. The intrabody placements of the distal end portions of the catheters 10, 20 can be carried out using an imaging modality such as ultrasound and/or fluoroscopy, or, as needed a laproscopy, as is well known. Typically, the first catheter 10 is placed using ultrasound-guided procedures while the magnet 15m at the distal end portion can be placed using fluoroscopy. The second catheter 20 can also be placed using fluoroscopy. As shown in FIG. 2, the inflatable tissue spacers 30b of the second catheter 20 can be expanded (either before or as the window 40 approaches the magnet/window of the first catheter 10) on the other side of the tissue. When the magnet 15m is configured to be in the desired location and in alignment with the other magnet 15m/window 40, the tissue spacers 30b can be deflated, which allows the catheter body to move closer the wall and positions the window 40 closer to the target tissue. The magnet 15m can be deployed to form a compressive lock against the other magnet 15m on the other side of the tissue. If a clinician/surgeon decides that the placement is not optimum or proper, the spacers 30b can be re-inflated and the window repositioned. If the magnet 15m has been deployed out of the window, then it may be pulled back in via string 62. In some embodiments, the magnetic force can be measured with an onboard sensor 135 (FIG. 7) if, for example, the gall bladder wall is too thick (as determined by a reduced force), the magnet 15 may not be deployed, and/or an alert can be generated so that a clinician can make appropriate adjustments. Once deployed, the magnets 15m generate magnetic compression on the tissue and can form an anastomosis over time. The magnets 15m are subsequently passed through the formed opening into the duodenum and out of the body via the intestinal system (e.g., in stool).

In some embodiments, the catheter 10 in the gall bladder can be configured to remain in the gall bladder over a course of time to drain the gall bladder (typically 1-2 weeks). To retain the catheter 10 in the gall bladder, it may have a “J-shape” also known as a “pig tail.” Also, when appropriate, the catheter 10 can be used to drain the gall bladder before the magnet 15m is deployed. Thus, the catheter 10 can include a drainage channel that is the same channel used to hold the mechanical pusher (where used) or a different channel.

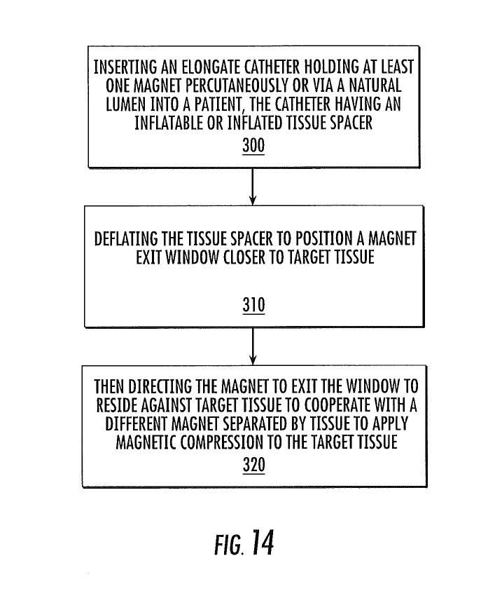

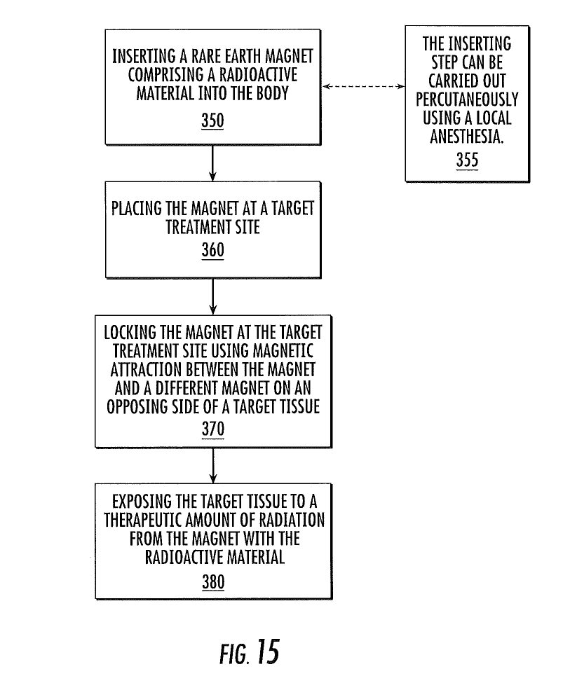

As shown in FIG. 14, according to some embodiments methods of placing intrabody magnets for magnetic compression of tissue therebetween can include: (a) inserting an elongate catheter holding at least one magnet percutaneously or via a natural lumen into a patient, the catheter having an inflatable or inflated tissue spacer (block 300); deflating the tissue spacer to position a magnet exit window closer to target tissue (block 310); then directing the magnet to exit the window to reside against target tissue to cooperate with a different magnet separated by tissue to apply magnetic compression to the target tissue (block 320).

As shown in FIG. 15, methods of treating intrabody tissue with radiation can include: (a) inserting a magnet (which may optionally be a rare earth magnet) comprising a radioactive material into the body (block 350); (b) placing the magnet at a target treatment site (block 360); (c) locking the magnet at the target treatment site using magnetic attraction between the placed magnet and a different magnet on an opposing side of target tissue (block 370); and (d) exposing the target tissue to a therapeutic amount of radiation from the magnet with the radioactive material (block 380). The inserting step may be carried out percutaneously or via a natural lumen (block 355) avoiding the need for invasive laparotomy.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. In the claims, means-plus-function clauses, if used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A catheter, comprising:
an elongate flexible catheter body;
at least one magnet held in the catheter body;
a window on a distal end portion of the catheter body configured to allow the magnet to exit the catheter body; and
at least one tissue spacer extending out from the catheter body proximate the window, wherein the tissue spacer is configured to retract or deflate from an extended or inflated configuration.

2. The catheter of claim 1, wherein the catheter body has an inner channel and an outer wall enclosing the inner channel, wherein the window is a longitudinally extending window extending through the outer wall having opposing first and second ends extending a defined length of the catheter body, and wherein the at least one tissue spacer comprises a first tissue spacer proximate the first end of the window and a second tissue spacer proximate the second end of the window.

3. The catheter of claim 1, wherein the at least one tissue spacer is an inflatable tissue spacer.

4. The catheter of claim 3, wherein the at least one tissue spacer comprises a first inflatable tissue spacer proximate one end of the window and a second inflatable tissue spacer proximate an opposing longitudinal end of the window.

5. The catheter of claim 1, wherein the at least one magnet is a rare earth magnet.

6. The catheter of claim 5, wherein the rare earth magnet comprises neodymium.

7. The catheter of claim 1, wherein the magnet comprises a radioactive material.

8. The catheter of claim 7, wherein the radioactive material comprises a beta emitter having a half-life that is between about 24 to about 120 hours.

9. The catheter of claim 7, wherein the radioactive material comprises Yttrium-90.

10. The catheter of claim 1, further comprising a pusher configured to reside in the elongate catheter body and move axially and/or laterally to force the magnet out the window.

11. The catheter of claim 1, further comprising a magnetic force sensor on the catheter body proximate the window.

12. A delivery system for placing intrabody magnets for generating magnetic compression, the system comprising:
a first catheter having a magnet held therein for delivery to a target intrabody site, the first catheter having at least one inflatable tissue spacer thereon; and
a second catheter having a magnet held therein for delivery to a target intrabody site;
wherein, the first and second catheters deliver respective cooperating magnets to generate magnetic compression on target tissue.

13. The delivery system of claim 12, further comprising:
at least one conduit attached to the first catheter in fluid communication with the at least one inflatable tissue spacer.

14. The delivery system of claim 13, further comprising a control circuit in communication with the first catheter configured to carry out at least one of the following: (a) monitor magnetic force data sensed by a sensor on the first and/or second catheters; (b) control the inflation and deflation of the at least one inflatable tissue spacer thereon; or (c) generate an alert when magnetic force is detected to be insufficient and/or sufficient to thereby confirm appropriate placement.

15. The delivery system of claim 12, wherein the first catheter comprises a magnet exit window and spaced apart first and second inflatable tissue spacers proximate each longitudinal end of the window.

16. The delivery system of claim 12, wherein at least one of the magnets of the first and second catheter comprises a radioactive material.

17. A surgical device, comprising:
a rare earth magnet comprising a therapeutic agent held in a sterile package for surgical use.

18. The surgical device of claim 17, wherein the therapeutic agent comprises a radioactive material.

19. The surgical device of claim 17, further comprising a catheter that is configured to hold the rare earth magnet for intrabody placement.

20. The surgical device of claim 18, wherein the radioactive material comprises a beta emitter, and wherein the magnet further comprises a string attached to the magnet.

21. A method of placing intrabody magnets for magnetic compression of tissue therebetween, comprising:
inserting an elongate catheter holding at least one magnet percutaneously or via a natural lumen into a patient, the catheter having an inflatable or inflated tissue spacer;
deflating the tissue spacer to position a magnet exit window closer to target tissue; and
then directing the magnet to exit the window to reside against target tissue to cooperate with a different magnet separated by tissue to apply magnetic compression to the target tissue.

22. A method of treating intrabody tissue with radiation, comprising:
inserting a magnet comprising a radioactive material into the body;
placing the magnet at a target treatment site;
compressing tissue at the target treatment site using magnetic attraction between the placed magnet and a different magnet on an opposing side of tissue at the target treatment site; and
exposing the tissue to a therapeutic amount of radiation from the magnet with the radioactive material.
